(19) United States
(12) Patent Application Publication
(10) Pub. No.: US 2013/0302368 A1
(43) Pub. Date: Nov. 14, 2013

(54) Advanced Prime and Boost Vaccine

(71) Applicant: Boro Dropulic, Ellicott City, MD (US)

(72) Inventor: Boro Dropulic, Ellicott City, MD (US)

(21) Appl. No.: 13/981,173

(22) PCT Filed: Jan. 27, 2012
(86) PCT No.: PCT/US12/23015

§ 371 Date: Jul. 26, 2013

Related U.S. Application Data
(60) Provisional application No. 61/436,828, filed on Jan. 27, 2011.

(51) Int. Cl. A61K 39/145; A61K 39/02; A61K 39/00; A61K 39/12

(52) U.S. Cl. CPC A61K 39/145; A61K 39/12; A61K 39/02; A61K 39/0011
USPC 424/199.1; 424/204.1; 424/209.1; 424/218.1; 424/234.1

(57) ABSTRACT

This invention relates to vaccines and in particular to the combination of non-integrating, replication-incompetent retroviral vectors (NIV) with virus-like particle (VLP) vaccines to induce an immune response in an animal host following administration to the host. This combination results in a novel vaccine strategy for delivering priming and boost doses, wherein an effective amount of an NIV is administered to the host, followed by an effective amount of a VLP. The concept can be broadly applied to infectious disease vaccines and also to cancer vaccines.

### CROSS-REFERENCE TO RELATED PATENT APPLICATIONS

This application claims the benefit of and priority to U.S. Provisional Patent Application No. 61/436,828, filed Jan. 27, 2011, which is incorporated herein by reference in its entirety.

### FIELD OF THE INVENTION

This invention relates to vaccines and in particular to the combination of non-integrating, replication-incompetent retroviral vectors (NIV) with virus-like particle (VLP) vaccines to induce an immune response in an animal host following administration to the host. This combination results in a novel vaccine strategy for delivering priming and boost doses. The concept can be broadly applied to infectious disease vaccines (e.g. Dengue, Malaria, Hepatitis C, etc.) and also to cancer vaccines.

### BACKGROUND

Retroviruses are enveloped RNA viruses that belong to the family Retrovirida. After infecting a host cell, the RNA is transcribed into DNA via the enzyme reverse transcriptase. The DNA is then incorporated into the cell's genome by an integrase enzyme and thereafter replicates as part of the host cell's DNA. The Retrovirida family includes the genera Alpharetrovirus, Betaretrovirus, Gammaretrovirus, Deltaretrovirus, Epsilonretrovirus, Lentivirus, and Spumavirus.

Retroviral vectors are well-known to persons skilled in the art. They are enveloped virion particles derived from retroviruses that are infectious but non-replicating. They contain one or more expressible polynucleotide sequences. Thus, they are capable of penetrating a target host cell and carrying the expressible sequence(s) into the cell, where they are expressed. Because they are engineered to be non-replicating, the transduced cells do not produce additional vectors or infectious retroviruses.

Retroviral vectors derived from Gammaretroviruses are well known to the art and have been used for many years to deliver genes to cells. Such vectors include ones constructed from murine leukemia viruses, such as Moloney murine leukemia virus, or feline leukemia viruses.

Lentiviral vectors derived from Lentiviruses are also well known to the art. They have an advantage over retroviral vectors in being able to integrate their genome into the genome of non-dividing cells. Lentiviruses include human immunodeficiency virus (HIV), simian immunodeficiency virus (SIV), bovine immunodeficiency virus (BIV), equine infectious anemia virus, feline immunodeficiency virus, puma lentivirus, caprine arthritis encephalitis virus, and visna/maedi virus.

These vectors, being foreign antigens, produce an immune response in an animal host. The present invention uses this response to create a desirable immunity in an animal host.

### DESCRIPTION OF THE INVENTION

The invention relates to a method for vaccinating a host, comprising a first step of administering an effective amount of an NIV to the host and a second step of administering an effective amount of a VLP to the host. The NIVs transduce cells in the host and the transduced cells produce VLPs.

The NIV comprises a non-integrating, non-replicating retroviral vector comprising a long terminal repeat, a packaging sequence, and a heterologous promoter operably linked to one or more polynucleotide sequences that together encode the structural proteins of a virus. The structural proteins self-assemble into a VLP when the polynucleotide sequences are expressed in a cell transduced by the vector. In a preferred embodiment, the retroviral vector is a lentiviral vector.

The NIVs of the invention act as self-boosting vaccines. The particle not only acts as a vaccine itself, but it also produces antigenic VLPs after entering the cells, since it encodes for VLP production from its non-integrating genome. This provides a second round of immune stimulation.

The VLPs produced by the transduced cells may be the same as or different from the VLPs administered in the second step. In a preferred embodiment, the VLPs are the same. Thus, the second step provides a boost to the immunity created by the first step, in effect providing a third round of immune stimulation.

The administration of NIVs results in an initial exogenous MHC Class II presentation of the antigen, and then the continuous production of VLPs from NIVs results in endogenous MHC Class I presentation of antigen through the steady release of small amounts of VLPs from transduced cells. Then, boosting at a later date with VLPs provides a larger dose of exogenous antigen to drive rapid expansion of already primed reactive clones responsive to the MHC Class II presentation of antigen.

As used herein, the term “an effective amount” means an amount sufficient to cause an immune response in the mammal or other animal host. Such amount can be determined by persons skilled in the art, given the teachings contained herein.

The two-step vaccine, also called a prime and boost vaccine, can be directed to any infectious disease. In one embodiment, the infectious disease is a viral disease. In one aspect, the viral disease is influenza, dengue fever, CMV, or West Nile fever. In another aspect, the infectious disease is a bacterial disease. Examples are tuberculosis infection, staphylococcus aureus infection, and pseudomonas aeruginosa infection.

The vaccine can also be directed to cancer by targeting cancer specific antigens. Examples of such antigens include Her-2, Muc1, BCR-ABL, and other cancer antigens that are known in the art.

The host can be any animal. Preferably, it is a mammal. In one embodiment, the mammal is a laboratory animal. For example, it can be a rodent, such as a mouse, rat, or guinea pig, or a dog, cat, or non-human primate. In another embodiment, the mammal is a human.

The NIV's and VLPs can be delivered by any known vaccine delivery system. In one embodiment, they are delivered subcutaneously. In another embodiment, they are delivered intramuscularly. The NIVs and VLPs in each step can be delivered by different methods or the same method.

VLPs are not viruses. They consist only of an outer viral shell and do not have any viral genetic material. They do not contain full-length genomic viral RNA. Thus, they do not replicate. The expression of capsid proteins of many viruses leads to their spontaneous assembly into supramolecular, highly repetitive, icosohedral or rod-like particles similar to the native virus they are derived from but free of viral genetic material. Thus, VLPs represent a non-replicating, non-infectious particle antigen delivery system that stimulates both native and adaptive immune responses. Being particulate, they provide the critical “danger signal” that is important for the generation of a potent and durable (after multiple immunizations) immune response. VLPs can be extremely diverse in terms of the structure, consisting of single or multiple capsid proteins either with or without lipid envelopes and with or without non-lipid envelopes. The simplest VLPs are non-enveloped and assemble by expression of just one major capsid protein, as shown for VLPs derived from hepadnaviruses, papillomaviruses, parvoviruses, or polyomaviruses.

NIVs are similar to VLPs, except that they also contain genetic information that can express the proteins after they enter a cell. In this invention, the NIVs express viral proteins comprising VLPs after entry into cells. Therefore, not only is the NIV itself a VLP-like vaccine (having a core and antigens in a particle), but upon entry into cells after administration to the host animal, the viral genetic information efficiently enters the nucleus without integration. Here it expresses to high levels proteins that are then assembled to make VLP particles inside the body, amplifying the immunogenic effect. This results not only in a strong primary immune response but a persistent one that can generate long lasting immunity.

A further advantage of NIV vaccines is the small amount needed to generate an immune response. Since the particles are amplified after being produced from cells in the body, the amount of initial material needed to generate an immune response is very small, dramatically improving the economics of such a vaccine.

The NIV of the invention comprises a non-integrating, non-replicating retroviral vector comprising a long terminal repeat, a packaging sequence, and a heterologous promoter operably linked to one or more polynucleotide sequences that together encode the structural proteins of a virus. In one embodiment, the retroviral vector is a gammaretroviral vector. In another embodiment, it is a lentiviral vector. In one aspect of this embodiment, the lentiviral vector is an HIV vector or an SIV vector. For example, it can be a non-integrating, non-replicating lentiviral vector comprising HIV long terminal repeats, an HIV packaging sequence, and a heterologous promoter operably linked to an HIV gag gene. Such a vector may further comprise an HIV env gene and an HIV pol gene that comprises a mutated integrase sequence that does not encode a functional integrase protein. In one particular aspect, it is an HIV-1 vector. In any of these embodiments and aspects, it may be a self-inactivating (SIN) vector. For example, it can be a non-integrating, non-replicating HIV SIN vector with an inactivating deletion in the U3 region of the 3′ LTR comprising an HIV LTR, an HIV packaging sequence, and a heterologous promoter operably linked to an HIV gag sequence and an HIV pol sequence, wherein the pol sequence comprises an integrase sequence that does not encode a functional integrase protein.

As mentioned above, the NIV comprises a heterologous promoter operably linked to one or more polynucleotide sequences that together encode the structural proteins of a virus. This causes the transduced cells to produce immune-stimulating VLPs. The virus can be any virus to which immunity is desired. These include viruses from the following families: Adenoviridae, Arenaviridae, Astroviridae, Baculoviridae, Bunyaviridae, Calciviridae, Coronaviridae, Filoviridae, Flaviridae, Hependnaviridae, Herpesviridae, Orthomyoviridae, Paramyxoviridae, Parvoviridae, Papovaviridae, Picornaviridae, Poxviridae, Reoviridae, Retroviridae, Rhabdoviridae, and Togaviridae. Examples include lentivirus, influenza virus, hepatitis virus, alphavirus, filovirus, and flavivirus. More specific examples include HIV-1, SIV, Influenza A virus, Influenza B virus, Hepatitis C virus, Ebola virus, Marburg virus, CMV, and Dengue Fever virus.

In a further embodiment of the invention, the NIV includes a heterologous polynucleotide that codes for polypeptide that is not a structural protein of a virus. In one embodiment, this protein is an antigen. The antigen can be any protein or part thereof. It can be derived from a virus, bacteria, parasite, or other pathogen. Such antigens are well-known in the art. In one embodiment, the antigen is a tumor antigen. In one aspect of this embodiment, the tumor antigen is a cell membrane protein.

In another embodiment, the heterologous protein is an immunomodulating protein. An immunomodulating protein is any protein that is involved in immune system regulation or has an effect upon modulating the immune response. In one embodiment, it is a cytokine, such as an interleukin, an interferon, or a tumor necrosis factor. In one aspect, the cytokine is IL-2, IL-12, GM-CSF, or G-CSF. Other cytokine examples that modulate the immune response that could be incorporated are found at www.ncbi.nlm.nih.gov. Such examples are incorporated herein by reference in their entireties. Immunomodulating protein are not restricted to cytokines They can be other proteins, such as ligands or protein fragments that act as ligands. They can also be comprised of antibodies that target ligand binding sites on target proteins on cells. One example of antibodies and ligands are CTLA-4 antibodies and the CD-40L protein. Other examples are found in the art and some can be found at www.ncbi.nlm.nih.gov. Such examples are incorporated herein by reference in their entireties.

The NIVs of the invention are constructed by techniques known to those skilled in the art, given the teachings contained herein. Techniques for the production of retroviral vectors are disclosed in U.S. Pat. Nos. 4,405,712, 4,650,746, 4,861,719, 5,672,510, 5,686,279, and 6,051,427, the disclosures of which are incorporated herein by reference in their entireties. Techniques for the production of lentiviral vectors are disclosed in U.S. patent application Ser. No. 11/884,639, published as US 2008/0254008 A1, and in U.S. Pat. Nos. 5,994,136, 6,013,516, 6,165,782, 6,294,165 B1, 6,428,953 B1, 6,797,512 B1, 6,863,884 B2, 6,924,144 B2, 7,083,981 B2, and 7,250,299 B1, the disclosures of which are incorporated herein by reference in their entireties.

The invention includes plasmids, helper constructs, packaging cells, and producer cells used to construct and produce the NIVs. The plasmid comprises retroviral long terminal repeat sequences, a retroviral packaging sequence, and a heterologous promoter operably linked to one or more polynucleotide sequences that together encode the structural proteins of a virus. In one embodiment, the retroviral sequences are lentiviral sequences. In one aspect of this embodiment, the lentiviral sequences are HIV sequences. The packaging cell comprises the plasmid of the invention and a helper construct that does not contain an integrase gene or contains an integrase gene that is not functional. In one embodiment, the cell is a mammalian cell. The producer cell comprises the plasmid of the invention and a helper construct that does not contain an integrase gene or contains an integrase gene that is not functional. In one embodiment, the cell is a mammalian cell. The producer cells can be used to produce the VLPs administered in the second step of the method of the invention.

The VLPs of the invention comprise structural proteins of a target virus. The virus is any virus for which the vectors of the invention can produce self-assembling structural proteins that form a VLP. Examples are described above. The proteins may be limited to capsid proteins of a particular virus, or they could also include envelope proteins of the same virus. The capsid and envelope proteins can be from the same or different viruses. In one embodiment, the VLP includes a heterologous envelope protein, such as a VSV-G envelope protein, influenza A virus envelope protein, influenza B virus envelope protein, hepatitis C virus envelope protein, Ebola virus envelope protein, Marburg virus envelope protein, or dengue fever virus envelope protein. In another embodiment, the VLP includes a heterologous protein that is an antigen or an immunomodulating protein as described above. The VLPs are produced by techniques known to those skilled in the art, given the teachings contained herein.

As mentioned above, the NIVs can include a heterologous polynucleotide sequence that encodes an antigen or an immunomodulating protein. In such case, the VLPs will include the antigen or immunomodulating protein.

The antigen can be any protein or part thereof. It can be derived from a virus, bacteria, parasite, or other pathogen. It can also be a tumor antigen, such as a cell membrane protein from a neoplastic cell. It can also be a tumor antigen that is not on the cell membrane. In such cases, such tumor antigens are either incorporated with transmembrane domains, so that they are expressed on the surface of the particles, or they are singly expressed within the cell without linkage to any other protein. The tumor antigens can also be linked to other protein or peptide sequences that increase the immunogenicity of the tumor antigen. Such sequences are known in the art and they generally stimulate native immunity through TLR pathways.

Additional information about NIVs and VLPs is found in international application number PCT/US2010/027262, filed Mar. 13, 2010, and published on Sep. 16, 2010 as WO 2010/105251 A2 and the corresponding US national phase application number 13/256,216, both of which are incorporated herein by reference in their entireties.

The invention includes pharmaceutical compositions comprising the NIVs of the invention and a pharmaceutically acceptable carrier or the VLPs of the invention and a pharmaceutically acceptable carrier. Such carriers are known to those skilled in the art and can be determined from the teachings contained herein. For example, the carrier can be an isotonic buffer that comprises lactose, sucrose, or trehalose.

In addition, the pharmaceutical compositions can include an adjuvant. Such adjuvants are known to those skilled in the art and can be determined from the teachings contained herein. For example, they include one or more of alum, lipid, water, buffer, peptide, polynucleotide, polymer, or an oil.

The invention further includes a kit for vaccinating a mammal. The kit comprises the pharmaceutical compositions of the invention and containers for them. The kit can further include instructions for use of the compositions.

The benefits of this invention are multiple: (1) Class I and Class II antigenic stimulation pathways are utilized, using the combined NIV-VLP prime-boost vaccine strategy, providing a high potential for generation of potent and durable protective immune responses; (2) inherent flexibility of the lentiviral vector system easily can accommodate multiple sub-types to produce a broadly reactive vaccine; (3) lentiviral-based NIV prime vaccine expresses Dengue E protein like a DNA vaccine but in context of a VLP; optionally, it can additionally express cytokines or RNAi to enhance immune response; (4) VLP's stimulate both innate and adaptive immunity, permitting multiple boosting of immune response with high levels of antigen to drive rapid expansion of the NIV-primed reactive clones. One of the significant advantages of this invention is the ability to produce NIV and VLP vaccines for prime and boost using a single integrative platform.

The following examples illustrate certain aspects of the invention and should not be construed as limiting the scope thereof.

### EXAMPLES

### Example 1: Creation of a Novel Dengue Fever Virus Vaccine

This example describes the creation of a novel Dengue fever virus vaccine capable of offering protection against multiple strains of the Dengue virus. The two-component vaccine has a priming dose comprised of a non-integrating vector (NIV) vaccine that is a virus-like particle (VLP) itself but contains non-integrating genomes that encode for production of VLPs from transduced cells. The vaccine is designed to incorporate the epitopes of the E protein from a series of isolates of each subtype of the Dengue virus, which are combined into one antigen that shares common elements from all of the isolates.

The second component of the vaccine is a boost with similar VLPs that lack genetic material and, as a result, do not themselves produce additional VLPs as do the NIVs.

The administration of NIVs results in an initial exogenous MHC Class II presentation of the Dengue antigen and then the continuous production of VLPs from NIVs results in endogenous MHC Class I presentation of antigen through the steady release of small amounts of VLPs from transduced cells. Then boosting at a later date with VLPs provides a larger dose of antigen to drive rapid expansion of already primed reactive clones responsive to the MHC Class II presentation of antigen.

The NIVs and VLPs can be manufactured using skills known in the art, given the teachings contained herein. The VLPs used for boosting the immune response will be produced from cell lines that are transduced with vectors that are similar to NIV vectors, but are capable of integrating to enable stable cell line generation.

While the NIV vaccine encodes for proteins that generate VLPs upon cell transduction in vivo, the boosting VLP does not contain any genetic information, permitting effective use of the boosting vaccine for multiple administrations, if necessary.

The vaccine can be developed as follows:

1. Construct and characterize the vaccine—Non-integrating vectors will be used for development of the NIV vectors. Integrating versions of these vectors will be used for generating cell lines that produce VLPs. Four final constructs will be developed (one for each of the 4 sub-types) for animal studies after in vitro characterization.

2. Process development & vaccine manufacture—After the animal material is manufactured, process optimization will continue in preparation for future clinical trials.

3. Perform mouse immunogenicity studies—VLPs will be tested for immunogenicity in mice. Two rounds of studies are planned to optimize vaccine composition and dose. (NIVs can only be tested in non-human primates.)

4. Perform monkey immunogenicity and challenge studies—Combined NIV prime and VLP boost vaccines will be tested in non-human primates.

5. Human clinical trials—these will be done to show the ability to generate the desired immune response.

### Example 2: Creation of VLPs

Many infectious disease antigens have been identified. In a similar manner to what has been described in Example 1, after antigens have been identified from various infectious diseases, they can be incorporated into the NIV and also into integrating versions based on the NIV in order to produce NIVs and VLPs for the prime and boost vaccination steps.

For example, the VLPs could comprise the structural proteins of a virus. The virus is any virus for which the vectors of the invention can produce self-assembling structural proteins that form a VLP. These include lentiviruses, other retroviruses, influenza viruses, hepatitis viruses, filoviruses, flaviviruses or any of the virus derived from families described above in this application. In particular embodiments, the viruses are selected from the group consisting of HIV-1, SIV, Seasonal and Pandemic Influenza, including Influenza A virus and Influenza B virus strains, Hepatitis A, B or C virus, Arbovirus infections including West Nile Virus, Ebola virus, Cytomegalovirus, Respiratory Syncitial virus, Rabies virus, Corona virus infections, including SARS, Human Papilloma virus, Rotaviruses, Herpes Simples Virus, Marburg virus, and Dengue fever virus. The structural proteins comprise the core of the virus. They can also include the envelope of the virus.

### Example 3: Antigens

As a further example, the VLPs could comprise any infectious disease antigen or cancer antigen wherein antigenic epitopes of these antigens are fused to envelope proteins of the VLP so as to present the antigen of interest on the surface of a VLP. The antigen can be any protein or part thereof. It can be derived from a virus, bacteria, parasite, or other pathogen. It can also be a tumor antigen, such as a cell membrane protein from a neoplastic cell. It can also be a tumor antigen that is not on the cell membrane. In such cases, such tumor antigens are either incorporated with transmembrane domains, so that they are expressed on the surface of the particles, or they are singly expressed within the cell without linkage to any other protein. The tumor antigens can also be linked to other protein or peptide sequences that increase the immunogenicity of the tumor antigen. Such sequences are known in the art and they generally stimulate native immunity through TLR pathways.

As an example, the epitope of a cancer or infectious disease agent could be fused to the hemagglutinin protein of an influenza virus VLP. Many specific broad cancer antigens have been identified. In a similar manner to what has been described in Example 1, after antigens have been identified from various cancers, they can be incorporated into the NIV and also into integrating versions based on the NIV in order to produce NIVs and VLPs for the prime and boost vaccination steps.

Although this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

### REFERENCES

All publications, including issued patents and published patent applications, and all database entries identified by url addresses or accession numbers are incorporated herein by reference in their entirety.

1. A method for vaccinating a mammal comprising a first step of administering an effective amount of a non-integrating, non-replicating lentiviral vector, comprising a long terminal repeat, a packaging sequence, and a heterologous promoter operably linked to one or more polynucleotide sequences that together encode the structural proteins of a virus, to the mammal, wherein the structural proteins self-assemble into a VLP when the polynucleotide sequences are expressed in a cell transduced by the vector and a second step of administering an effective amount of a VLP to the mammal.

2. (canceled)

3. (canceled)

4. The method of claim 1 wherein the VLP administered in the second step is the same as the VLP produced by the transduced cells in the mammal after the administration of the NIV.

5. The method of claim 4 wherein the method creates an immune response in the mammal to an infectious disease.

6. The method of claim 5 wherein the infectious disease is a viral disease.

7. The method of claim 6 wherein the viral disease is selected from the group consisting of influenza, dengue fever, and West Nile fever.

8. The method of claim 5 wherein the infectious disease is a bacterial disease.

9. The method of claim 4 wherein the method creates an immune response in the mammal to a cancer in the mammal.

10. (canceled)

11. The method of claim 1 wherein the mammal is a human.

12. The method of claim 1 wherein the vector comprises a self-inactivating (SIN) vector.

13. The method of claim 1 wherein the lentiviral vector is an HIV vector.

14. (canceled)

15. The method of claim 1 wherein the virus is selected from the group consisting of lentivirus, influenza virus, hepatitis virus, alphavirus, filovirus, and flavivirus.

16. (canceled)

17. The method of claim 1 wherein the structural proteins comprise the capsid of the virus.

18. The method of claim 17 wherein the structural proteins further include the envelope of the virus.

19. The method of claim 1 wherein the vector comprises a heterologous polynucleotide sequence that codes for a heterologous protein.

20. The method of claim 19 wherein the heterologous protein is a heterologous envelope protein.

21. The method of claim 19 wherein the heterologous protein is selected from the group consisting of an antigen and an immunomodulating protein.

22. (canceled)

23. The method of claim 1 wherein the vector is pseudotyped with a heterologous envelope protein.

24. The method of claim 23 wherein the heterologous envelope protein is selected from the group consisting of a VSV-G envelope protein, influenza A virus envelope protein, influenza B virus envelope protein, hepatitis C virus envelope protein, Ebola virus envelope protein, Marburg virus envelope protein, and dengue fever virus envelope protein.

25-29. (canceled)

30. The method claim 1 wherein the VLP further comprises a heterologous polypeptide selected from the group consisting of an antigen and an immunomodulating protein.

31. The method of claim 30 wherein the antigen is a tumor antigen.

32. (canceled)

33. (canceled)
